(19) United States
(12) Patent Application Publication
(10) Pub. No.: US 2021/0177596 A1
(43) Pub. Date: Jun. 17, 2021

(54) ANNULOPLASTY IMPLANT

(71) Applicant: Medtentia International Ltd Oy, Espoo (FI)

(72) Inventors: Hans-Reinhard ZERKOWSKI, Kreuzlingen (CH); Olli KERÄNEN, Bjärred (SE)

(21) Appl. No.: 16/758,611

(22) PCT Filed: Oct. 26, 2018
(86) PCT No.: PCT/EP2018/079518

§ 371 Date: Apr. 23, 2020

Related U.S. Application Data
(60) Provisional application No. 62/578,095, filed on Oct. 27, 2017.

(51) Int. Cl. A61F 2/24

(52) U.S. Cl. CPC A61F 2/2445; A61F 2230/0091; A61F 2220/0033; A61F 2220/0016

(57) ABSTRACT

An annuloplasty implant is disclosed comprising first and second support rings being adapted to be arranged as a coil in a coiled configuration around an axial direction, wherein the first and second support rings are adapted to be arranged on opposite sides of native heart valve leaflets of a heart valve to pinch said leaflets, fastening units fixed to the first support ring, wherein the fastening units are configured for interlocking with the second support ring via deformation of the fastening units when in the coiled configuration. A method of manufacturing an annuloplasty implant is disclosed, and a method of repairing a defective heart valve.

### FIELD OF THE INVENTION

This invention pertains in general to the field of cardiac valve replacement and repair. More particularly the invention relates to an annuloplasty implant, such as an annuloplasty ring or helix, for positioning at the heart valve annulus, a method of manufacturing an annuloplasty implant, and a method of repairing a defective heart valve.

### BACKGROUND OF THE INVENTION

Diseased mitral and tricuspid valves frequently need replacement or repair. The mitral and tricuspid valve leaflets or supporting chordae may degenerate and weaken or the annulus may dilate leading to valve leak. Mitral and tricuspid valve replacement and repair are frequently performed with aid of an annuloplasty ring, used to reduce the diameter of the annulus, or modify the geometry of the annulus in any other way, or aid as a generally supporting structure during the valve replacement or repair procedure. The annuloplasty ring is typically implanted around the annulus of the heart valve.

A problem with prior art annuloplasty implants is to achieve correct positioning at the heart valve and fixate the implant in the correct position. Suturing devices for annuloplasty implants have disadvantages that makes it difficult to suture in the correct position, thereby resulting insufficient suturing strength, and also in a very time-consuming procedure, which increases the risks for the patient. Furthermore, suturing devices are often not sufficiently compact for catheter based procedures. The use of clips for positioning annuloplasty implants is also associated with challenges, in particular when implanting helix rings that are to be positioned on either side of a heart valve.

Insufficient fixation of such implant lead to traumatic effects since the fixation structure must ensure the correct position of the device over time. A further problem in the prior art is thus also to achieve a reliable fixation at the annulus of the heart valve. An annuloplasty implant is intended to function for years and years, so it is critical with long term stability in this regard.

The above problems may have dire consequences for the patient and the health care system. Patient risk is increased.

Hence, an improved annuloplasty implant would be advantageous and in particular allowing for avoiding more of the above mentioned problems and compromises, and in particular ensuring secure fixation of the annuloplasty implant, during the implantation phase, and for long-term functioning, in addition to a less complex procedure, and increased patient safety. A related method would also be advantageous.

### SUMMARY OF THE INVENTION

Accordingly, examples of the present invention preferably seek to mitigate, alleviate or eliminate one or more deficiencies, disadvantages or issues in the art, such as the above-identified, singly or in any combination by providing a device according to the appended patent claims.

According to a first aspect an annuloplasty implant is provided comprising first and second support rings being adapted to be arranged as a coil in a coiled configuration around an axial direction, wherein the first and second support rings are adapted to be arranged on opposite sides of native heart valve leaflets of a heart valve to pinch said leaflets, fastening units fixed to the first support ring, wherein the fastening units are configured for interlocking with the second support ring via deformation of the fastening units when in the coiled configuration.

According to a second aspect a method of manufacturing an annuloplasty implant is provided comprising forming first and second support rings configured to be arranged in a coiled configuration around an axial direction, forming fastening units integrated into the first support ring, wherein the fastening units are configured for interlocking with the second support ring via deformation of the fastening units when in the coiled configuration.

According to a third aspect a method of repairing a defective heart valve is provided, comprising positioning first and second support rings of an annuloplasty implant in a coiled configuration on opposite sides of native heart valve leaflets of the heart valve, interlocking fastening units integrated into the first support ring with the second support ring by deforming the fastening units.

Further examples of the invention are defined in the dependent claims, wherein features for the second and subsequent aspects are as for the first aspect mutatis mutandis.

Some examples of the disclosure provide for a facilitated positioning of an annuloplasty implant at a heart valve.

Some examples of the disclosure provide for a facilitated fixation of an annuloplasty implant at a heart valve.

Some examples of the disclosure provide for a less time-consuming fixation of an annuloplasty to a target site.

Some examples of the disclosure provide for securing long-term functioning and position of an annuloplasty implant.

Some examples of the disclosure provide for a reduced risk of damaging the anatomy of the heart such as the annulus or the valve leaflets.

Some examples of the disclosure provide for facilitated manufacturing of an annuloplasty implant.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a schematic illustration of an annuloplasty implant according to an example of the disclosure;

FIG. 2 is a schematic illustration of an annuloplasty implant, when in a stretched elongated configuration, according to an example of the disclosure;

FIGS. 3a-b are detail views of a fastening unit and a corresponding recess in a support ring, when separated, according to examples of the disclosure;

FIG. 3c is a detail view of a fastening unit and a corresponding recess in a support ring, when in an interconnected configuration, according to an example of the disclosure;

FIG. 4a is a detail view of a fastening unit and a corresponding recess in a support ring, when separated, according to an example of the disclosure;

FIG. 4b is a detail view of a fastening unit and a corresponding recess in a support ring, when in an interconnected configuration, according to an example of the disclosure;

FIG. 5a is a detail view of a fastening unit and a corresponding recess in a support ring, when separated, according to an example of the disclosure;

FIG. 5b is a detail view of a fastening unit and a corresponding recess in a support ring, when in an interconnected configuration, according to an example of the disclosure;

FIG. 6a is a schematic illustration of an annuloplasty implant according to an example of the disclosure;

FIG. 6b is a schematic illustration of an annuloplasty implant, when in an implanted state, according to an example;

FIG. 7a is a flow chart of a method of manufacturing an annuloplasty implant according to an example;

FIG. 7b is another flow chart of a method of manufacturing an annuloplasty implant according to one example;

FIG. 8a is a flow chart of a method of repairing a defective heart valve according to an example; and

FIG. 8b is another flow chart of a method of repairing a defective heart valve according to one example.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The following description focuses on an embodiment of the present invention applicable to cardiac valve implants such as annuloplasty rings. However, it will be appreciated that the invention is not limited to this application but may be applied to many other annuloplasty implants and cardiac valve implants including for example replacement valves, and other medical implantable devices.

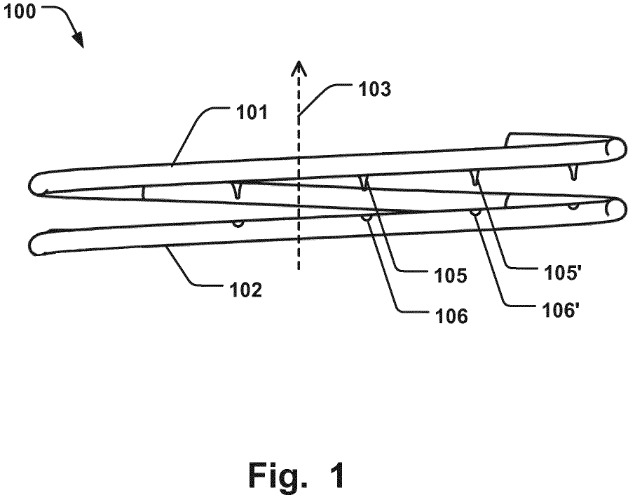

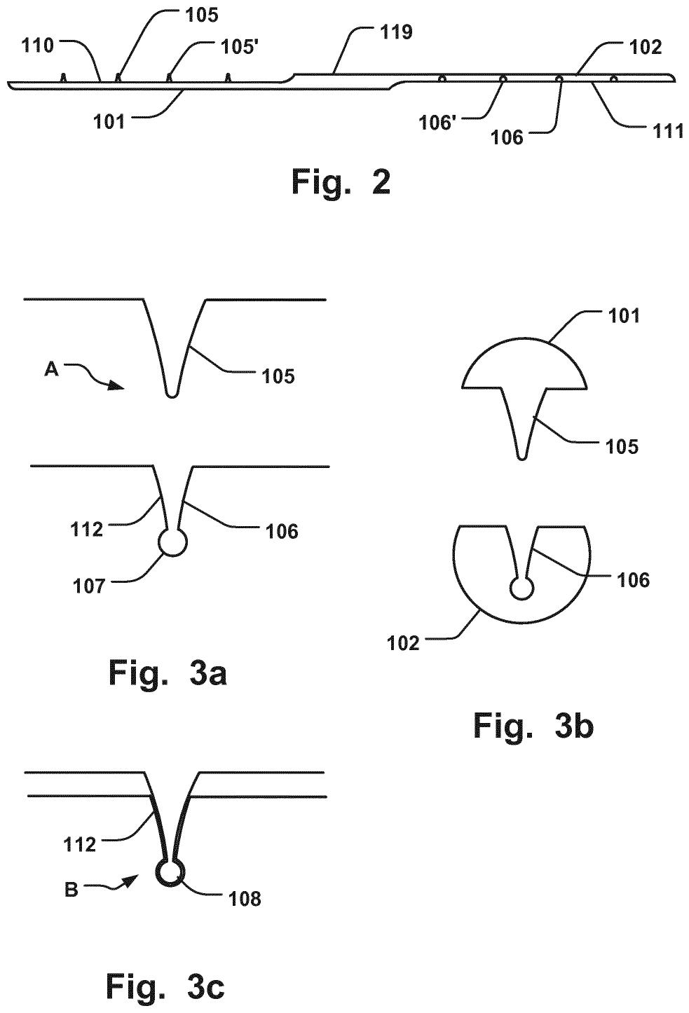

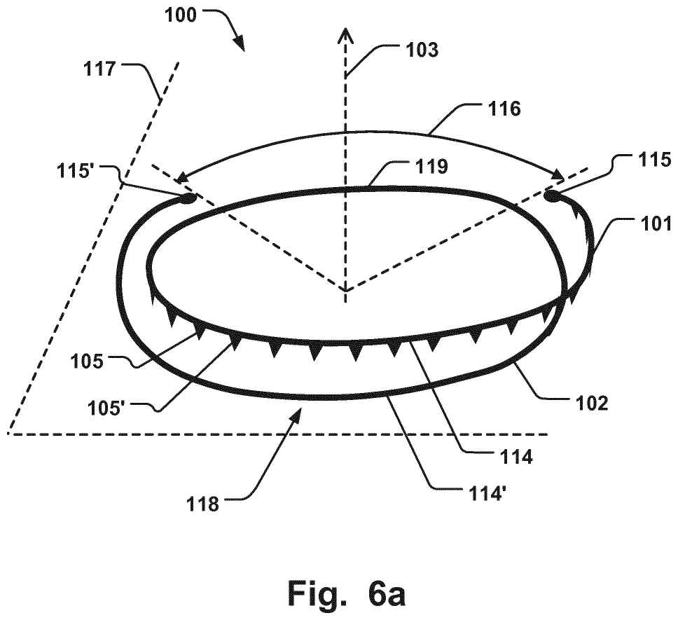

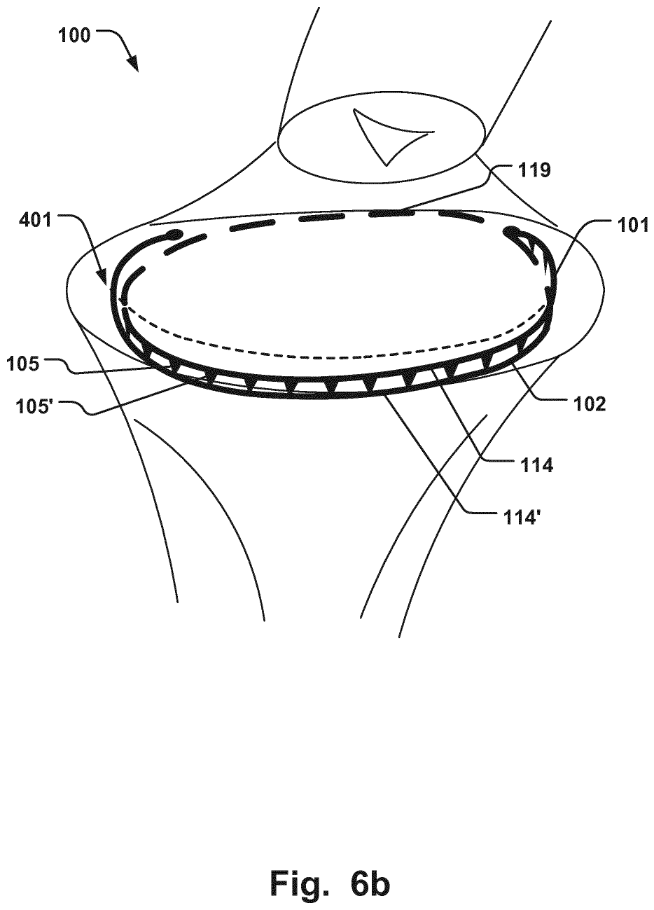

FIG. 1 is a schematic illustration of an annuloplasty implant 100 comprising first 101 and second 102 support rings being adapted to be arranged as a coil in a coiled configuration around an axial direction 103. The first and second support rings 101, 102, are adapted to be arranged on opposite sides of native heart valve leaflets of a heart valve, as illustrated in FIG. 6b. As shown in FIG. 6b, the first support 101 may be arranged on an atrial side of the valve, and the second support 102 may be arranged on a ventricular side. The first and second supports 101, 102, are connected to form a coil- or helix shaped ring. The coil extends through the valve opening (dashed line) at a commissure 401 thereof. In the examples of FIGS. 6a-b, the second support 102, positioned on the ventricular side, forms a complete loop, whereas the first support 101, positioned on the atrial side, has a reduced length along its periphery, as will be described further below. The implant 100 may comprise a shape-memory material, so that the implant 100 assumes the coiled configuration after having been ejected from a delivery catheter. While in the delivery catheter the implant 100 may be stretched in an elongated shape, i.e. as illustrated in FIG. 2. Alternatively, the implant 100 may be arranged in the coiled configuration when being delivered to the target site, in which case it may be implanted at the target site for example by incision between the ribs or by opening the chest. The present disclosure, and the associated advantages described for the various examples, applies to both such variants of the implant 100. The implant 100 comprises fastening units 105, 105′, fixed to the first support ring 101. The fastening units 105, 105′, are configured for interlocking with the second support ring 102 via deformation of the fastening units 105, 105′, when in the coiled configuration. FIG. 6b is a schematic illustration showing the fastening units 105, 105′, being interlocked with the second support 102. I.e. the first and second support rings 101, 102, are configured to be arranged on opposite sides of native heart valve leaflets of the heart valve to pinch the leaflets when the fastening units 105, 105′, are interlocked with the second support 102. Having fastening units 105, 105′, interlocking with the second support 102 by being deformed provides for a particularly strong and robust fixation of the annuloplasty implant 100 at the heart valve, since the deformation of the material of the fastening units 105, 105′, allows for said material to conform to the structure and shape of the second support 102, so that an interlocked connection can be formed therebetween, via mating surfaces of the fastening units 105, 105′, and the second support 102. This also provides for interlocking the first and second supports 101, 102, with a force fit connection, which does not necessitate cumbersome connection mechanisms, such as screws, sutures or the like, which require a lengthier and more precise manipulation by the operator in order to be fixed into position. Such precise manipulation is difficult to execute on the beating heart, due to the significant movement of the surrounding anatomy. The force-fit connection provided by the deformable fastening units 105, 105′, for interlocking with the second support 102 this allows for a quicker and safer procedure. Furthermore, since the fastening units 105, 105′, are fixed to the first support 101, the number of components that must be navigated to, and manipulated at, the target site can be minimized. I.e. suturing devices or other fixation elements or tools can be dispensed with.

Although the fastening units 105, 105′, are fixed to the first support 101 in the example in e.g. FIGS. 6a-b, it is conceivable that the fastening units 105, 105′, can be fixed to the second support 102 positioned on the ventricular side, for interlocking with the first support 101 at the atrial side. The number of fastening units 105, 105′, can also be varied to optimize the retention strength when interlocked with the second support 102.

The fastening units 105, 105′, may be integrated into the first support ring 101, e.g. by being formed from the material of the first support ring 101. This may provide for an enhanced structural integrity of the fastening units 105, 105′. The fastening units 105, 105′, may be hence cut from the material of the first support 101. FIG. 3a-b shows an example of a fastening units 105 being cut from the material of the first support 101. FIG. 3a show an example of an implant 100 in a stretched elongated configuration, and the different sections thereof. As mentioned, the fastening units 105, 105′, may be provided on the first support 101, and as described further below, the fastening units 105, 105′, may interlock with structures arranged on the second support 102 such as recesses 106, 106. An intermediate portion 119, without fastening units 105, 105′, may be positioned therebetween. The intermediate portion 119 may thus be positioned to be arranged at the anterior side when the implant 100 is attached to the heart valve, as schematically illustrated in FIG. 6b. The intermediate portion 119 may thus comprise a smooth surface free from fastening units 105, 105′. This may ensure that there is no risk of piercing the tissue at the anterior side of the valve. FIGS. 3a-c, 4a-b, and 5a-b are magnified views of sections of the implant 100 showing examples of the fastening units 105, 105′, and recesses 106.

In the case the fastening units 105, 105′, are formed from the material of the first support 101, the material may be removed from an initially substantially circular cross-section of the support 101 to create fastening units 105, 105′, as shown in the cross-section of FIG. 3b. The upper part of FIG. 3b, showing the fastening unit 105 thus corresponds to the cross-section of the first support 101 in FIG. 2, and the lower part of FIG. 3b shows an example of a cross-section of the second support 102 in FIG. 2, illustrating the recess 106 into which the fastening unit 105 may interlock. FIGS. 3a and 3c show magnified views in the lengthwise direction, i.e. as illustrated in FIG. 2, but when the second support 102 has been bent into a position opposite the first support 101 so that the fastening units 105, 105′, are arranged opposite the recesses 106, 106′.

The fastening units 105, 105′, may be cut to form various shapes to facilitate the deformation thereof when interlocking with the second support and optimizing the retention force when in the interlocked state. The fastening units 105, 105′, may be formed by different cutting techniques such as milling or laser cutting techniques. It is also conceivable that the fastening units 105, 105′, may be fixed or integrated onto the first support 101 by other methods, or by being formed from other materials. The support 101, 102, may be formed from a solid rod or other solid elongated structure, having various cross-sections, such as circular, elliptic, rhombic, triangular, rectangular etc. The support 101, 102, may be formed from a hollow tube, or other hollow structures with the mentioned cross-sections. The support 101, 102, may be formed from a sandwiched laminate material, comprising several layers of different materials, or different layers of the same material. The support 101, 102, may be formed from a stent or a stent-like structure, and/or a braided material. The support 101, 102, may be formed from a braid of different materials braided together, or from a braid of the same material. The support 101, 102, may be formed from NiTinol, or another suitable bio-compatible material. The surfaces of the first and second supports 101, 102, may be provided with other materials and/or treated with different materials and/or structured to enhance resistance to breaking in case the material is repeatedly bent.

Hence, the fastening units 105, 105′, may comprise extensions 105, 105′, configured to interlock with corresponding recesses 106, 106′, arranged in the second support ring 102, as exemplified in FIGS. 1-5. The recesses 106, 106′, have been omitted in FIGS. 6a-b for the purpose of clarity of presentation. FIGS. 3a-c, 4a-b, and 5a-b, show different examples of recesses 106 being shaped and configured to interlock with corresponding fastening units 105 which deforms when interlocking with the recesses 106. This provides for a robust force fit connection that allows for a safe and facilitated clamping of the support rings 101, 102, on the opposite sides of the valve leaflets for an efficient pinching thereof and remodeling of the valve annulus.

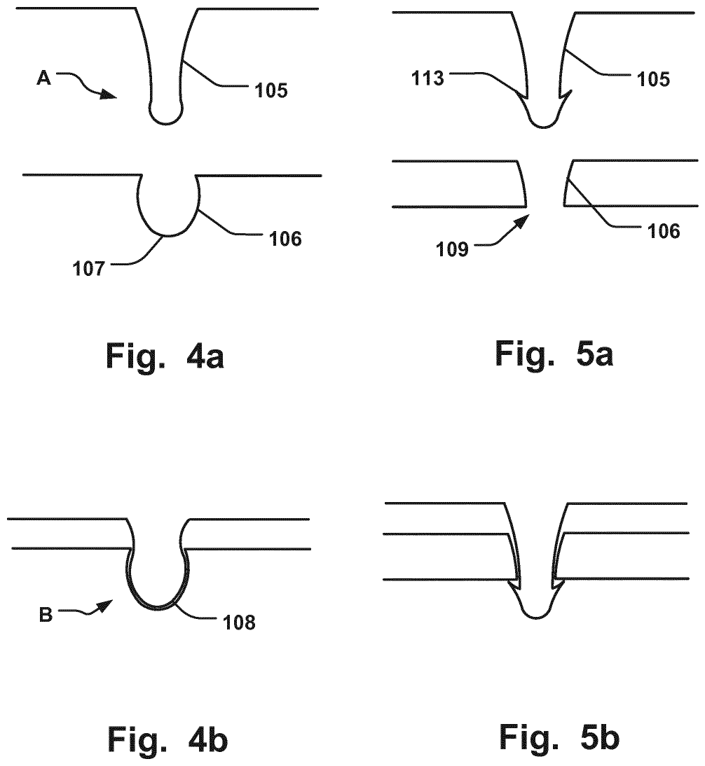

The fastening units 105, 105′, may be configured to be plastically deformed when interlocking with the second support ring 102. Plastically deforming the fastening units 105, 105′, allows for a strong irreversible connection to the second support 102. The fastening units 105, 105′, may comprise a material that is configured to irreversibly engage a corresponding mating surface 107 of the second support ring 102 for interlocking with the mating surface 107 in a locked state. FIG. 3a show an example where the fastening unit 105 has a tapered shape towards a distal tip thereof. FIG. 3b is a cross-section seen along a longitudinal extension of the support 101, showing the same tapered shape. In FIG. 3c, the tapered fastening unit 105 has been pushed into recess 106 so that the distal tip of the tapered shape as been deformed to fill up a void in the recess, defined by the mating surface 107. The fastening unit 105 may thus interlock with the recess 106 with the fastening principle of a rivet. FIGS. 4a-b show another example where the fastening unit 105 has an initial blunt distal tip (FIG. 4a), before being pushed into the recess 106 (FIG. 4b) to be deformed against the mating surface 107 and further compressed to assume a shape which is increasingly circular. In both examples, the recess 106 has a narrow neck portion at the entrance of the void of the recess 106. The deformed fastening unit 105 will thus deform and expand to fill a wider portion of a bottom of the void of the recess 106, so that the narrow neck portion at said entrance will provide a counter force against the deformed fastening unit 105.

The extensions, i.e. the fastening units 105, may have a delivery configuration (A) and a deformed configuration (B) in the locked state in which the extensions are deformed to at least partly fill corresponding recesses 106 in the second support ring 102 with a deformed portion 108 of a material of the fastening units 105, 105′, as illustrated in FIG. 3a, 3c, and FIGS. 4a-b. The recesses 106 thus comprise the mating surface 107. The deformed portion 108 may not fill the recess 106 completely as shown in the discussed example, but sufficiently to allow a counter force to act against the deformed fastening unit 105 as elucidated above.

The first support ring 101 may be configured to be clamped to the second support ring 102 so that the fastening units 105, 105′, are deformed for said interlocking. The clamping may be provided by a clamping tool that push the fastening units into the corresponding recesses with a sufficient force to deform the fastening units 105, 105′, in the recesses 106, 106′.

Although the discussed examples show recesses 106, 106′, for interlocking with the extensions of the fastening units 105, 105′, it is conceivable that the fastening units 105, 105′, may interlock with the second implant 102 in other ways. The fastening units 105, 105′, may e.g. be deformed to clamp onto the outside of the second support 102, thus allowing interlocking without recesses 106, 106′.

The recesses 106, 106′, may comprise through holes 109 in the second support ring 102, as shown in FIGS. 5a-b. The fastening units 105, 105′, may be configured to be elastically or plastically deformed when arranged to extend across the through holes 109 for interlocking with the second support 102. In FIG. 5b, an example is shown where the fastening unit 105 comprise a retention unit 113 configured to be elastically deformed when pushed into the through hole 109. The retention unit 113 is configured to be subsequently expanded for interlocking with the opposite side of the through hole 109 as illustrated. This provides for a secure interlocking with the second support 102.

The first and second support rings 101, 102, may comprise substantially flat opposite surfaces 110, 111, arranged against each other when the fastening units 105, 105′, are interlocked with the second support ring 102. The extensions of the fastening units 105, 105′, may thus extend from a first flat surface 110 of the first ring 101 towards corresponding recesses 106, 106′, formed in a second opposite surface 111 of the second support ring 102, as illustrated in e.g. FIGS. 2, 3a-c. This may provide for a compact cross-section of the implant 100 and further a facilitated interlocking due to the flat surfaces 110, 111, supporting each other.

The recesses 106, 106′, may comprise sloped surfaces 112 arranged so that the corresponding extensions can slide against the slope surfaces 112 when interlocking with the recesses 106, 106′. Thus, the fastening units 105, 105′, as shown in FIGS. 3a-b, may be guided against the slope surfaces 112 into the recesses 106, 106′, facilitating the interlocking.

The first support ring 101 may be adapted to be arranged on an atrial side of the heart valve, and the second support ring 102 may be adapted to be arranged on a ventricular side of the heart valve. The first support ring 101 may comprise a first posterior bow 114 and the second support ring 102 may comprise a second posterior bow 114′, as illustrated in FIG. 6a. The fastening units 105, 105′, may be arranged on the first posterior bow 114 for interlocking with the second posterior bow 114′, as illustrated in FIG. 6b. As mentioned above, this allows for a secure fixation of the implant 100 while minimizing risk of damaging tissue at the critical portions of the anatomy at the anterior side, at the anterior portion 119 of the implant 100.

The first and second support rings 101, 102, may have respective free ends 115, 115′, configured to be arranged on opposite sides of the native heart valve leaflets, when in the coiled configuration. The two free ends 115, 115′, may be displaced from each other with a peripheral off-set distance 116 extending in a coil plane 117, as illustrated in FIG. 6a. The coil plane 117 is substantially parallel to an annular periphery 118 of the coil and perpendicular to the central axis 103. The coil plane 117 accordingly corresponds to the plane spanned by the annular periphery 118 of the implant 100 when assuming the coiled configuration. The peripheral off-set distance 116 between the two free ends 115, 115′, thus extends substantially perpendicular to the central axis 103. This means that, when the implant 100 is positioned in the implanted state, around the annulus of the heart valve, the two free ends will be separated along the plane of the valve. By having such off-set 116 in the plane of the valve, the resulting reduced length of the first or second support member 101, 102, will allow for reducing the number of fastening units 105, 105′, required to securely fixate the implant 100 at the valve, while at the same time providing for a sufficient overlap of the first and second support member 101, 102, on the opposites sides of the valve to attain a sufficiently strong pinching effect therebetween to fixate the annulus in a modified shape. In situations, placing fastening units 105, 105′, on the anterior side may be associated with high risk, as discussed above. This can therefore be avoided, by having the off-set 116 as specified. Furthermore, the interference of the implant 100 with the movements of the valve will be minimized. Fastening of the implant 100 on the atrial side can thus be accomplished by fixation of the posterior bows 114, 114′, and there will be no interference on the atrial side with the movement of the valve, due to the off-set distance 116 reducing the circle sector of the first support 101.

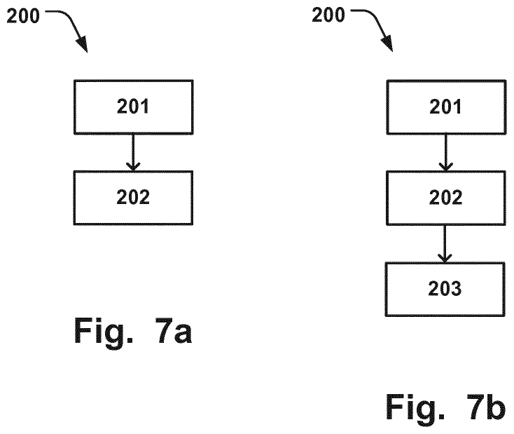

FIG. 7a illustrates a method 200 of manufacturing an annuloplasty implant 100. The order in which the steps of the method 200 are illustrated should not be construed as limiting and it is conceivable that the order in which the steps of the method 200 is carried out may be varied. The method 200 comprises forming 201 first 101 and second 102 support rings configured to be arranged in a coiled configuration around an axial direction 103, and forming 202 fastening units 105, 105′, integrated into the first support ring 101. The fastening units 105, 105′, are configured for interlocking with the second support ring 102 via deformation of the fastening units when in the coiled configuration. Thus, the method 200 thus provides for an annuloplasty implant 100 with the advantageous effects described above in relation to FIGS. 1-6.

FIG. 7b illustrates a further method 200 of manufacturing an annuloplasty implant 100. The order in which the steps of the method 200 are illustrated should not be construed as limiting and it is conceivable that the order in which the steps of the method 200 is carried out may be varied. The method 200 may comprise forming 203 recesses 106, 106′, integrated into the second support ring 102, being configured to interlock with the fastening units 105, 105′, when the implant 100 is in the coiled configuration.

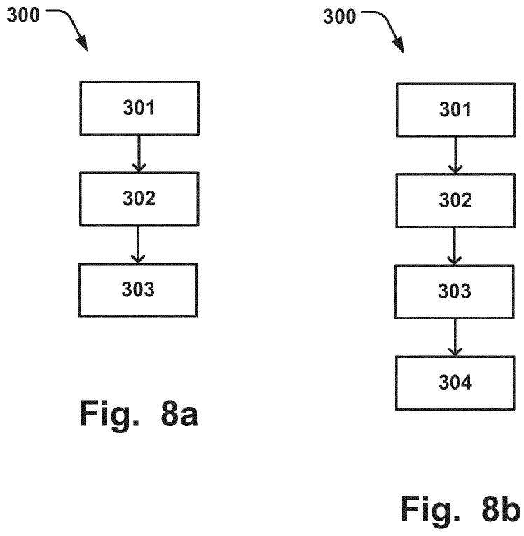

FIG. 8a illustrates a method 300 of repairing a defective heart valve. The order in which the steps of the method 300 are illustrated should not be construed as limiting and it is conceivable that the order in which the steps of the method 300 is carried out may be varied. The method 300 comprises positioning 301 first and second support rings 101, 102, of an annuloplasty implant 100 in a coiled configuration on opposite sides of native heart valve leaflets of the heart valve, and interlocking 302 fastening units 105, 105′, integrated into the first support ring 101 with the second support ring 102 by deforming 303 the fastening units 105, 105′. The method 300 thus provides for the advantageous effects described above in relation to FIGS. 1-6.

FIG. 8b illustrates a further method 300 of repairing a defective heart valve. The order in which the steps of the method 300 are illustrated should not be construed as limiting and it is conceivable that the order in which the steps of the method 300 is carried out may be varied. Deforming the fastening units 105, 105′, may comprise plastically deforming 304 extensions 105, 105′, of the fastening units so that a deformed portion 108 of a material of the fastening units 105, 15′, at least partly fill corresponding recesses 106, 106′, in the second support ring 102, arranged opposite the extensions. A secure and reliable fixation of the implant 100 at the heart valve is thus provided.

The present invention has been described above with reference to specific embodiments. However, other embodiments than the above described are equally possible within the scope of the invention. The different features and steps of the invention may be combined in other combinations than those described. The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

1. An annuloplasty implant comprising
first and second support rings being adapted to be arranged as a coil in a coiled configuration around an axial direction, wherein the first and second support rings are adapted to be arranged on opposite sides of native heart valve leaflets of a heart valve to pinch said leaflets,
fastening units fixed to the first support ring, wherein the fastening units are configured for interlocking with the second support ring via deformation of the fastening units when in the coiled configuration.

2. The annuloplasty implant according to claim 1, wherein the fastening units are integrated into the first support ring.

3. The annuloplasty implant according to claim 1, wherein the fastening units are formed from the material of the first support ring.

4. The annuloplasty implant according to claim 1, wherein the fastening units comprise extensions configured to interlock with corresponding recesses arranged in the second support ring.

5. The annuloplasty implant according to claim 1, wherein the fastening units are configured to be plastically deformed when interlocking with the second support ring.

6. The annuloplasty implant according to claim 5, wherein the fastening units comprise a material that is configured to irreversibly engage a corresponding mating surface of the second support ring for interlocking with the mating surface in a locked state.

7. The annuloplasty implant according to claim 4, wherein the extensions have a delivery configuration (A) and a deformed configuration (B) in the locked state in which the extensions are deformed to at least partly fill corresponding recesses in the second support ring with a deformed portion of a material of the fastening units, the recesses comprising said mating surface.

8. The annuloplasty implant according to claim 1, wherein the first support ring is configured to be clamped to the second support ring so that the fastening units are deformed for said interlocking.

9. The annuloplasty implant according to claim 4, wherein the recesses comprise through holes in the second support ring, and wherein the fastening units are configured to be elastically or plastically deformed when arranged to extend across the through holes for said interlocking.

10. The annuloplasty implant according to claim 9, wherein the fastening units comprise retention units configured to be elastically deformed when pushed into the through holes and to be subsequently expanded for said interlocking.

11. The annuloplasty implant according to claim 4, wherein the first and second support rings comprise substantially flat opposite surfaces arranged against each other when the fastening units are interlocked with the second support ring, whereby the extensions extend from a first flat surface of the first ring towards the corresponding recesses formed in a second opposite surface of the second support ring.

12. The annuloplasty implant according to claim 4, wherein the recesses comprise sloped surfaces arranged so that the corresponding extensions can slide against the slope surfaces when interlocking with the recesses.

13. The annuloplasty implant according to claim 1, wherein the first support ring is adapted to be arranged on an atrial side of said heart valve, and the second support ring is adapted to be arranged on a ventricular side of the heart valve,
wherein the first support ring comprises a first posterior bow and the second support ring comprises a second posterior bow and
wherein the fastening units are arranged on the first posterior bow for interlocking with the second posterior bow.

14. The annuloplasty implant according to claim 1, wherein the first and second support rings have respective free ends configured to be arranged on opposite sides of the native heart valve leaflets, when in said coiled configuration, whereby the two free ends are displaced from each other with a peripheral off-set distance extending in a coil plane, said coil plane being substantially parallel to an annular periphery of said coil and perpendicular to said central axis.

15. A method of manufacturing an annuloplasty implant comprising;
forming first and second support rings configured to be arranged in a coiled configuration around an axial direction, and
forming fastening units integrated into the first support ring, wherein the fastening units are configured for interlocking with the second support ring via deformation of the fastening units when in the coiled configuration.

16. The method according to claim 15, comprising
forming recesses integrated into the second support ring, being configured to interlock with the fastening units when the implant is in the coiled configuration.

17. A method of repairing a defective heart valve, comprising
positioning first and second support rings of an annuloplasty implant in a coiled configuration on opposite sides of native heart valve leaflets of the heart valve,
interlocking fastening units integrated into the first support ring with the second support ring by deforming the fastening units.

18. The method according to claim 17, wherein deforming the fastening units comprises
plastically deforming extensions of the fastening units so that a deformed portion of a material of the fastening units at least partly fill corresponding recesses in the second support ring, arranged opposite the extensions.
